(19) United States
(12) Patent Application Publication
(10) Pub. No.: US 2017/0355950 A1
(43) Pub. Date: Dec. 14, 2017

(54) SYSTEMS FOR DISSOCIATION OF BIOLOGICAL TISSUES

(71) Applicant: THE ARIZONA BOARD OF REGENTS ON BEHALF OF THE UNIVERSITY OF ARIZONA, TUCSON, AZ (US)

(72) Inventors: Yitshak ZOHAR, Tucson, AZ (US); Linda L. RESTIFO, Tucson, AZ (US); Linan JIANG, Tucson, AZ (US)

(21) Appl. No.: 15/532,854

(22) PCT Filed: Dec. 4, 2015
(86) PCT No.: PCT/US15/63978

§ 371 Date: Jun. 2, 2017

Related U.S. Application Data
(60) Provisional application No. 62/087,471, filed on Dec. 4, 2014.

(51) Int. Cl. C12M 1/33; B01L 3/00; C12M 3/06; C12M 1/34; C12N 5/0793

(52) U.S. Cl. CPC C12M 45/02; C12M 41/46; C12N 5/0619; C12M 23/16; B01L 3/50273; B01L 3/502746; B01L 2300/023; B01L 2400/0478; C12N 2509/10; B01L 2200/0605; B01L 2300/0681

(57) ABSTRACT

Provided herein is technology relating to processing biological samples and particularly, but not exclusively, to systems and apparatuses for dissociating biological tissues into viable cells.

The present application claims priority to U.S. Provisional Patent Application Ser. No. 62/087,471, filed Dec. 4, 2014, the disclosure of which is herein incorporated by reference in its entirety.

### STATEMENT REGARDING FEDERALLY SPONSORED RESEARCH OR DEVELOPMENT

This invention was made with government support under Grant No. R21 NS055774 awarded by NIH. The government has certain rights in the invention.

### FIELD

Provided herein is technology relating to processing biological samples and particularly, but not exclusively, to systems and apparatuses for dissociating biological tissues into viable cells.

### BACKGROUND

Primary neuron culture is a powerful technique. Cultured neurons manifest morphological and physiological phenotypes due to normal developmental transitions, mutations, and exposures to drugs and toxins (Kraft, et al., J Neurosci, 26(34): 8734-47, 2006). However, the method is not suitable for large-scale projects based on high-throughput assays. Worse, it is still more of an art than a science. For almost 50 years, the method has relied on mechanical dissociation of developing brain tissue by manual trituration, which involves repeated flushing of microdissected, enzyme-treated tissue pieces through a narrow pipette tip, which breaks the cells apart from each other and from their neurites (axons and dendrites) (Higgins and Banker, Primary dissociated cell cultures. In Culturing Nerve Cells, G. Banker and K. Goslin, Editors. 1998, The MIT Press: Cambridge, Mass. p. 37-78). The shear stress applied during trituration has never been measured—let alone optimized—for brain tissue from any species. Thus, it is not surprising that the method is difficult to teach and troubleshoot, yields inconsistent numbers of viable neurons, and places the operator at risk for repetitive motion-related injury. Neuroscientists have dealt with this problem by designing small-scale experiments and improving technology ‘around’ neuron culture, such as new genetic markers, optimizing substrates and trophic factors, use of intracellular dyes and multi-electrode recording arrays, and new image analysis software (Halterman, et al., J Neurosci Methods, 177(2): 348-54, 2009; Heck, et al., J Neurosci Methods, 183(2): 202-12, 2009; Mok, et al., J Neurosci Methods, 179(2): 284-91, 2009; Park, et al., Biomed Microdevices, 11(6): 1145-53, 2009; Mooney, et al., Tissue Eng Part A, 16(5): 1607-19, 2010; Previtera, et al., J Biosci Bioeng, 2010). But none of the more than 2,400 neuron-culture research publications in the past decade reports an attempt to improve or replace manual dissociation. Methods developed for larger tissue samples are not suitable for small pieces of developing brain tissue (DeLorenzo, et al., Int Rev Neurobiol, 81: 59-84, 2007; Chen and Herrup, Rev Neurosci, 19(4-5): 317-26, 2008; Chen, et al., J Neurotrauma, 26(6): 861-76, 2009). Specifically, for small tissue samples, commercially available automated micropipetting devices increase bubble formation (which traps cells), cannot self-adjust as the dissociation proceeds, and severely limit real-time monitoring. Accordingly, new solutions are needed.

### SUMMARY

Provided herein is technology relating to processing biological samples and particularly, but not exclusively, to systems and apparatuses for dissociating biological tissues into viable cells suitable for diverse assays particularly, but not exclusively, to in vitro culture.

Accordingly, provided herein is technology related to a microfluidic device that enables controlled exposure of tissue to a flow-induced stress field, which induces dissociation of individual cells while allowing continuous video recording, direct observation, and quantification of flow and force parameters. As a key step in proof of concept, experiments have demonstrated that cells (e.g., neurons, e.g., from brain tissue) dissociated in the device are viable and capable of extending an arbor of neurites typical of neurons cultured after manual trituration.

In some embodiments, the system comprises a plurality of components (FIG. 1): e.g., (i) a microdevice, (ii) an external fluid handling system, including a programmable syringe pump and connectors, and (iii) a monitoring system (e.g., a sensor, e.g., an imaging sensor, e.g., a CCD camera) connected to a computer through a compound optical microscope for video recording. During operation, enzyme-treated tissue is loaded into the device and these components generate a flow-induced shear stress within the microchannel, within which the dissociation can be directly observed. Once the device design (configuration and dimensions) and fabrication are optimized for particular soft tissues, the system is amenable to automated operation with a high degree of control and, hence, improved consistency, as well as improved yield of viable cells. Studies have been carried out with tissue (e.g., brain tissue) from the model organism Drosophila melanogaster (“fruit fly”) because of its powerful genetic tools, but the technology can be readily adapted to vertebrate brain tissue or any other (e.g., non-neural) biological soft tissue.

The key features of such a microfluidic device (FIG. 2) include one or more orifices along a dissociation channel, and an array of such dissociation channels (FIG. 2A). The configuration of the dissociation channel (FIG. 2B) allows flow to exert sufficient stress (FIG. 2C) on the tissue sample (FIG. 2D) to induce cell separation. In the description of the microdevices herein, the channels, orifices, and other features have a length, width, and height (e.g., corresponding to coordinates in the x, y, and z directions, respectively) (See FIG. 2B). The length generally refers to the dimension along which flow (bulk flow) generally occurs (e.g., even though localized eddies, backflow, turbulence, etc. may occur along other dimensions); the height generally refers to the dimension measured relative to a solid support or base of the microdevice; the width generally refers to the remaining dimension.

Because brain tissue is soft and highly compliant, the sample can be deformed and squeezed through medium-sized passages under hydrodynamic loading and still remain intact. Therefore, in an exemplary embodiment the main channel nominal dimensions are approximately 300 μm in height and 1 mm in width allowing tissue loading and transfer. In some embodiments (FIG. 2E), the entire microdevice is a transparent microfluidic device approximately 40 mm long and 2 mm wide. The length of the microchannels is chosen to allow the establishment of the required flow field. The orifice gap size (width) in each device ranges between 200 μm and 10 μm, whereas the length various between 400 μm to 50 μm (FIG. 2B, 2C, 2E). In contrast with a uni-directional flow approach, this configuration allows manipulation of a bi-directional oscillating flow field along the channel during the dissociation process. Subjecting the tissue to periodic mechanical loading, due to its oscillations through the orifices, significantly lowers the stress level required for dissociation, thus increasing the yield of viable cells.

Embodiments of the technology provide a microfluidic device comprising a microchannel comprising orifices of various widths in a sequence decreasing from approximately 200 μm to approximately 20 μm along a single microchannel (FIG. 3). As the tissue dissociates into progressively smaller fragments, the fragments are forced through orifices of decreasing size. Thus, the tissue fragments experience gradually increasing shear forces that produce single cells downstream of the smallest orifice.

Additional embodiments provide a standardized, efficient, high-yield brain-tissue dissociation method, system, and apparatus. Some embodiments of the technology provide an integrated microfluidic system, providing a platform for, e.g., enzymatic pre-treatment, stress field application for dissociation, filtration-based separation of the neurons from neuropil debris, followed by placement of the viable neurons into culture chambers. By replicating the basic microfluidic unit, the platform is expanded in some embodiments to a multiplex format (e.g., for processing multiple samples in parallel, e.g., 1 to 10,000 samples, e.g., in a multi-well format, e.g., in particular configurations comprising 6, 24, 96, 384, 1536, 3456, 9600, etc. samples) allowing further integration with liquid robotic handling, environmentally controlled incubation for in vitro culture, fixation and immunostaining, and automated microscopy systems for cell visualization and image acquisition.

In some embodiments, the technology comprises hardware components, software components, and consumable components (e.g., single-use, sterile microdevices with a limited shelf life; culture medium; enzymes) as described herein. The technology finds use, for example, in individual labs conducting neuroscience or cell biology research, as well as in institutional core laboratory facilities that provide specialized equipment and expertise. Further, the technology finds use in research institutions, biotechnology companies, and pharmaceutical companies, e.g., in the fields of central nervous system research and drug discovery. Further, the technology is useful in both public and private sectors, e.g., for developmental neurotoxicology testing.

Accordingly, provided herein is technology related to a device and/or system for dissociating an input tissue sample into an output sample comprising viable cells, the system comprising a microfluidic device comprising an orifice configured to provide shear stress on a tissue sample; and a programmable pump configured to move the tissue sample cyclically through the orifice according to a set of fluid-flow parameters. In some embodiments, the system further comprises a sensor component configured to obtain data describing the dissociation of the tissue sample into viable cells. In some embodiments, the sensor component is an imaging component (e.g., microscopy). In some embodiments, the sensor component collects image data.

The technology is not limited in the type of sample that is processed. For example, in some embodiments the sample comprises a tissue (e.g., a neural tissue (e.g., a brain tissue) or a non-neural tissue (e.g., cardiac, gastrointestinal, pancreatic, or liver tissue)).

In some embodiments, the flow parameters are associated with a sample type (e.g., the flow parameters have been previously determined empirically to be appropriate for the efficient dissociation of the sample type). Accordingly, in some embodiments the flow parameters are established for the sample type prior to use of the system by a user. In some embodiments, the flow parameters are configurable by a user prior to or during dissociation of the tissue. Further, in some embodiments the flow parameters are monitored and adjusted by software or a user in real time. In some embodiments, the system further comprises a user interface to accept input from a user. For example, in some embodiments the input comprises a sample type. In some embodiments the input comprises flow parameters or the input (e.g., a sample type) is used to determine or select flow parameters. In some embodiments, the system comprises software configured to receive data from the sensor component and provide the flow parameters to the syringe pump.

The system is configured to operate according to a number of flow parameters, e.g., in some embodiments the flow parameters comprise one or more values of a flow rate through the microfluidic device, a shear stress, an oscillation frequency of flow through the microfluidic device, and/or a number of cycles of flow through the microfluidic device.

In exemplary embodiments, the flow parameters comprise one or more values of a flow rate through the microfluidic device of from approximately 40 μl/s to approximately 80 μl/s, an oscillation frequency of flow through the microfluidic device of from approximately 4 Hz to approximately 5 Hz, and a number of cycles of flow through the microfluidic device of from approximately 1 to 100 cycles. In exemplary embodiments, the flow parameters comprise one or more of a flow rate through the microfluidic device to provide a shear stress from approximately 10 to 10⁴ dyne/cm², an oscillation frequency of flow through the microfluidic device of from approximately 1 Hz to approximately 10 Hz, and a number of cycles of flow through the microfluidic device of from approximately 1 to 5000 cycles. Further, in some embodiments the flow parameters comprise a waveform describing the flow rate through the orifice as a function of time (e.g., a square wave, triangular wave, a sine wave, etc.).

The system provides a pulsed shear stress (e.g., a pulsed load) on the cells as they pass repeatedly through the orifice. The pulsed load (stress) on the sample minimizes or avoids damage on the cells that would otherwise occur if subjected to a sustained stress. Accordingly, in some embodiments the system is configured to exert a peak load on the cells during each cycle for a duration of from 0.050 to 0.50 seconds. In some embodiments, the pulsed load is provided by flow through the orifice that is a bi-directional oscillating flow. In summary, embodiments provide a system that provides a cyclic load on the tissue, e.g., to produce one or more stress gradient(s) on the tissue.

In some embodiments, the sample is an in vivo-grown tissue or an in vitro-grown tissue. In some embodiments, the sample has a weight from approximately 0.1 μg to approximately 1 mg. In some embodiments, the sample has a largest dimension that is approximately 50 μm to approximately 2 mm.

Some embodiments relate to optical monitoring of the sample in the microfluidic device. Accordingly, in some embodiments the microfluidic device is optically transparent.

The technology is not limited in the design and configuration of the microfluidic device, the channel(s) of the microfluidic device, and/or the orifice of the microfluidic device. For example, in some embodiments the microfluidic device comprises a single flow path comprising one orifice. In some embodiments, the orifice has a width of from 10 μm to 200 μm, a length of from 10 μm to 200 μm, and a height of from 200 to 500 μm. In some embodiments, the orifice has a width of from 10 μm to 500 μm, a length of from 10 μm to 1000 μm, and a height of from 200 to 500 μm. In some embodiments, the microfluidic device comprises a channel that has a length of from 20 mm to 50 mm, a width of from 500 μm to 2 mm, and a height of from 50 μm to 500 μm. In some embodiments, the orifice width is approximately 1% to approximately 10% of the channel width. In some embodiments, the orifice cross-sectional area is approximately 5% to 30% of the sample maximum cross-sectional area. In some embodiments, the microfluidic device comprises a channel having a cross-sectional area that is approximately 3 to 5 times of the sample maximum cross-sectional area. In some embodiments, the microfluidic device comprises a channel and the orifice cross-sectional area is approximately 1% to 10% of the channel cross-sectional area. In some embodiments, the microfluidic device comprises a channel and the width of the flow path decreases from the channel width to the orifice width over a length of approximately 5 μm to 15 μm. In some embodiments, the microfluidic device comprises a channel and the width of the flow path increases from the orifice width to the channel width over a length of approximately 5 μm to 1000 μm. In some embodiments, the width of the flow path increases from the orifice width to the channel width at an angle of approximately 60 degree with the walls being symmetric to the channel central axis.

Some embodiments further comprise an apparatus comprising the programmable syringe pump, the sensor component, and an interface to accept the microfluidic device. In some embodiments, the apparatus comprises a microprocessor. In some embodiments, the apparatus comprises reagents. In some embodiments, the apparatus interface provides mechanical and/or fluidic communication between the microfluidic device and the apparatus.

In some embodiments, the microfluidic device is consumable. In some embodiments, the microfluidic device is sterile.

In some embodiments, the microfluidic device comprises a filter (e.g., to separate the intact dissociated cells from subcellular fragments or other tissue debris).

In some embodiments, the system further comprises an enzyme for promoting the dissociation of the tissue sample into viable cells, e.g., in some embodiments the system comprises an enzyme that is a collagenase and/or a protease.

In some embodiments, the system provides flow in a closed-loop system.

In some embodiments, the flow stress at the orifice is approximately 10 to 500 dyne/cm². In some embodiments, the flow stress at the orifice is approximately 10 to 10⁴ dyne/cm². In some embodiments, a system is provided wherein the shear stress from cycle to cycle is substantially and/or effectively consistent. Accordingly, in some embodiments of the system, a first shear stress provided on the tissue sample by the microfluidic device at cycle n is within 20% of a second shear stress provided on the device at cycle n+1. In some embodiments, a first shear stress provided on the tissue sample by the microfluidic device at cycle n is within 10% of a second shear stress provided on the device at cycle n+1. And, in some embodiments, a first shear stress provided on the tissue sample by the microfluidic device at cycle n is within 5% of a second shear stress provided on the device at cycle n+1.

The system dissociates the cells of a tissue to provide an output sample comprising dissociated cells. In some embodiments, 50%, 60%, 70%, 80%, 90%, 95% or more of the cells in the tissue in the input sample are provided as dissociated cells in the output sample. Accordingly, in some embodiments the system produces an output sample comprising at least 50% of the cells of the input tissue sample in a dissociated state; in some embodiments, the system produces an output sample comprising at least 70% of the cells of the input tissue sample in a dissociated state; in some embodiments, the system produces an output sample comprising at least 90% of the cells of the input tissue sample in a dissociated state; in some embodiments, the system produces an output sample comprising at least 95% of the cells of the input tissue sample in a dissociated state.

In some embodiments, systems and methods described herein generate an output sample comprising viable cells. In some embodiments, at least 20% (e.g., at least 30%, 40%, 60%, 60%, 70%, 80%, 90%, 95%, or 99%) of cells are viable. In some embodiments, viable cells are able to survive and differentiate in 2-dimensional or 3-dimensional culture.

The technology also provides for the multiplexed processing of multiple samples in parallel (e.g., two or more samples). Accordingly, the technology relates to any of the system described herein configured as a multiplex system, e.g., a multiplex system comprising a plurality of microfluidic devices and/or wherein a microfluidic device comprises a plurality of channels each comprising an orifice. In some embodiments, the multiplex system is configured to process 2 to 100 samples in parallel. In some embodiments, the multiplex system is configured to process 2 to 1000 samples in parallel.

Related embodiments provide methods, e.g., a method for dissociating an input tissue sample into an output sample comprising dissociated viable cells, the method comprising providing a system according to any preceding claim; providing a set of flow parameters to the programmable pump; and providing an input sample for dissociation by the system. Some embodiments relate to imparting different shear stresses (loads) on the tissue samples during the course of dissociation, e.g., in some embodiments a higher shear stress is first applied (e.g., to break up larger clumps of cells into smaller clumps and to dissociate some cells), which is followed by a lower shear stress later (e.g., to dissociate additional cells). Accordingly, in some embodiments, the methods comprise providing a first set of flow parameters to provide a first shear stress on the input tissue sample during cycles 1 to n and providing a second set of flow parameters to provide a second shear stress on the input tissue sample during cycles n+1 to m.

In some embodiments, the methods provided are used to process a sample comprising a soft tissue, e.g., a neural tissue such as a brain tissue or a non-neural tissue (e.g., cardiac, gastrointestinal, pancreatic, or liver tissue). In some embodiments, the sample has a weight from approximately 0.1 μg to approximately 1 mg. In some embodiments, the sample has a largest dimension that is approximately 50 μm to approximately 2 mm. In some embodiments, the methods further comprise monitoring and/or adjusting the flow parameters. Further embodiments comprise collecting an output sample comprising viable cells. In some embodiments, the cells are cultured.

Additional embodiments provide a population of cells dissociated by the methods described herein, wherein at least 20% (e.g., at least 30%, 40%, 60%, 60%, 70%, 80%, 90%, 95%, or 99%) of the cells are viable.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a conceptual schematic showing a system embodiment comprising a flow-control system, a microscopy/imaging system through which a tissue sample dissociation in microfluidic device can be monitored and imaged, and one or more computers for operating the flow controls and camera, as well as for data acquisition, storage, and analysis.

FIG. 2 shows exemplary microdevices. FIG. 2A is a drawing showing exemplary configurations of a microdevice comprising a channel with one orifice and another comprising a channel with four orifices. A microdevice with an array of dissociation channels is also shown. This is a top-down view perpendicular to the x-y plane. FIG. 2B is a drawing showing an orifice and a surrounding portion of a channel with associated length, width, and height noted in the x, y, and z directions, respectively. FIG. 2C shows a portion of an orifice in a graphically displayed flow model based on mathematical simulation. The relative level of flow-induced shear stress in the channel is indicated by the color scale from black (high) to white (low). FIG. 2D is a photograph showing a brain tissue (specifically, the entire central nervous system of a larval Drosophila) after loading into a microfluidic device, and located adjacent to an orifice, according to an embodiment of the technology provided herein. The view is perpendicular to the x-y plane. FIG. 2E are photographs of a single-channel microdevice and a twin-channel microdevice, and photographs of orifices of two microdevices, as examples, configured according to an embodiment of the technology provided herein. The view is perpendicular to the x-y plane. Ink is filled in the channels for contrast of the microfluidic features in transparent microdevices.

FIG. 3 shows a drawing of an embodiment of a microdevice having a channel (white) comprising a plurality of orifices with decreasing sizes. A brain tissue, like that in FIG. 2D, that was loaded into the device (to the left, out of the area of view) and moved by oscillating flow. The tissue initially breaks up into clumps of cells, which are reduced in size by moving through progressively smaller orifices, and ultimately into single cells. (Note that the drawings of individual cells are not to scale; they are drawn considerably larger than they would be in typical soft tissues, in order to show diagrammatically the process of dissociation through the length of the microchannel). In this embodiment, the cells are recovered to the right of the drawing, out of the area of view, for primary 2-dimensional or 3-dimensional culture.

FIG. 4 is a graph showing qualitative results of brain-tissue dissociation, cell recovery, and neurite outgrowth after 1, 2, and 3 days in vitro (div) culture by two independent operators (“1” and “2”) using the system described herein. Outcomes after using the microsystem technology described are compared with outcomes after using the existing method (e.g., manual trituration with a pipette tip, “M”). Evaluation of degree of tissue dissociation, recovery of neurons, and neurite outgrowth after 1, 2, and 3 div is graded on a scale of 0, 1, 2, 3, 4, and 5, indicating “poor”, “fair”, “good”, “very good”, “excellent”, and “outstanding”, respectively. The results shown are mean performance of 22 microdevice trials by operator 1, 30 microdevice trials by operator 2, compared with 8 trials by manual trituration.

FIG. 5 compares neurite-outgrowth data for genetic-control (CASK Ex33) brain neurons from device vs. manual dissociation.

FIG. 6A compares neurite-outgrowth data for genetic-control (CASK Ex33) and mutant (CASK Δ18; two independent replicates) brain neurons cultured after device dissociation. The data show consistency between the two mutant replicates and significant differences (smaller neurite arbors with higher branch density) between the mutant and genetic-control neurons. This confirms previous studies performed solely by manual dissociation. FIG. 6B adds data (far right box plots) from a manual dissociation of mutant (CASK Δ18) brain neurons and compares the neurite-outgrowth parameters to the pair of device-dissociated mutant brains. Neurons dissociated in the device grow larger arbors, indicative of better cellular health.

FIG. 7 compares neurite-outgrowth data for mutant (CASK Δ18) brain neurons from (A) a single-channel device vs. a twin-channel device and (B) a single-channel device vs. a twin-channel device vs. manual dissociation.

FIG. 8 shows photomicrographs of rat E18 hippocampal tissue (A) in a microfluidic device, at the beginning of the dissociation (top) and after dissociation (bottom); and (B) device-dissociated neuronal clusters cultured for 4 days at high density.

FIG. 9 shows a photomicrograph of individual rat E18 hippocampal neurons cultured for 4 days after complete dissociation in a microfluidic device.

FIG. 10 shows a photomicrograph of heart muscle cells in a culture dish immediately after dissociation in a device.

FIG. 11 shows a photomicrograph of heart muscle cells cultured for one day after dissociation in a device.

FIG. 12 shows representative genetic-control (CASK Ex33) Drosophila neurons cultured for 3 days after dissociation by manual vs. microfluidic device methods, and immunofluorescently stained for neuronal membranes.

FIG. 13 shows representative mutant Drosophila neurons (CASK Δ18) cultured for 3 days after dissociation by manual vs. microfluidic device methods, and immunofluorescently stained for neuronal membranes.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology related to a microfluidic system for the dissociation of biological tissues, including technology for fabricating microfluidic devices as described herein as one component of the systems provided.

From an engineering perspective, the major challenges faced in dissociating tissue into individual viable cells are: (i) the large range of scales, e.g., from a tissue size of ≧500 μm to a cell size of approximately 10 μm, (ii) the finesse required to apply sufficient external force to overcome intrinsic binding forces while minimizing damage to the cells, and (iii) the need for optically clear, bio-compatible materials to guarantee cell viability through the entire process while avoiding microbial contamination.

Further, today's primary cell culture methods are reminiscent of molecular biology in the late 20th century. Individual labs mastered DNA sequencing and RNA transcript analyses, but could only study small numbers of genes. In contrast, automated DNA sequencing and microarray-based expression profiling, genotyping, and epigenetic characterization have enabled genome-wide experiments that have revolutionized life sciences, from evolution to medical diagnostics. Similarly, automated tissue dissociation would represent a technological breakthrough, enabling high-throughput studies of cultured cells for basic and applied research. These would include genome-wide phenotyping screens using RNA-interference methods, chemical screens for cell-type-specific toxicology testing, and drug screens for potential therapeutics for devastating neurological diseases.

A microelectromechanical systems (MEMS) approach provides a technology suitable for addressing these challenges. For example, it has evolved to incorporate new polymer materials compatible with biological tissues and cells (Ng, et al., Journal of Micromechanics and Microengineering, 14(2): 247-255, 2004; Yu, et al., Journal of Microelectromechanical Systems, 14(6): 1386-1398, 2005; Chan, et al., Journal of Micromechanics and Microengineering, 16(4): 699-707, 2006; Lee, et al., Nanotechnology, 17(4): S29-S33, 2006; Lee, et al., Lab on a Chip, 6(8): 1080-1085, 2006; Jiang, et al., Journal of Microelectromechanical Systems, 17(6): 1495-1500, 2008; Cheung, et al., Lab on a Chip, 9(12): 1721-1731, 2009; Cheung, et al., Journal of Microelectromechanical Systems, 19(4): 752-763, 2010; Cheung, et al., Journal of Micromechanics and Microengineering, 21(5)2011; Gudipaty, et al., Microfluidics and Nanofluidics, 10(3): 661-669, 2011; Jiang, et al., Optics Express, 19(4): 3037-3043, 2011; Yetisen, et al. Journal of Micromechanics and Microengineering, 21(5)2011; Zheng, et al., Lab on a Chip, 11(19): 3269-3276, 2011; Zheng, et al., Lab on a Chip, 11(20): 3431-9, 2011). As a result, microfluidic systems have been fabricated for high-throughput studies of bacteria, yeast, nematode worms, and mammalian liver cells (Ingham, et al., Proc Natl Acad Sci USA, 104(46): 18217-18222, 2007; Lee, et al., BioTechniques, 44: 91-95, 2008; Rohde, et al., Proc Natl Acad Sci USA, 104(35): 13891-5, 2007; Khetani et al., Nat Biotechnol, 26(1): 120-6, 2008). In addition, microfluidic devices have been used to culture manually dissociated neurons and to induce cell disruption (lysis) to liberate the contents for biochemical studies (Morel, et al., Lab Chip, 9(7): 1011-3, 2009; Shin, et al., J Nanosci Nanotechnol, 9(12): 7330-5, 2009; Gobbels, et al., J Insect Physiol, 56(8): 1003-1009, 2010; Koester, et al., Lab Chip, 10(12): 1579-86, 2010; Wieringa, et al., J Neural Eng, 7(1): 16001, 2010; Park, et al., J Vis Exp, (31) 2009; Park, et al., Biotechnol J, 4(11): 1573-7, 2009; Xu, et al., ICBN 2004 (International Conference on Bioengineering and Nanotechnology 2004), 26-29, Singapore. 2004).

In some embodiments, the configuration and dimensions of the device are configurable. Some embodiments provide an integrated operating system comprising configurable hardware and other instrumentation (e.g., controls and connectors) and, in some embodiments, a software interface for automated operation.

In some embodiments, flow simulations are conducted to account for the presence of tissue in the microchannel, e.g., to predict the flow stress levels experienced by the tissue during dissociation. In some embodiments, computation of the flow-induced shear stress, dissociation experiments, and associated outcomes based on culturing the dissociated neurons are used to configure the system operating conditions (e.g., to determine flow parameters). In some embodiments, parallel culture experiments using neurons obtained by traditional manual trituration provide a quantitative control for comparison and evaluation of the yield, viability, and qualitative features of the cells dissociated utilizing the described microsystems technology.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein. The section headings used herein are for organizational purposes only and are not to be construed as limiting the described subject matter in any way.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.” As used herein, the phrase “microfluidic device”, “microdevice”, or “microfluidic cartridge” refers to a device, cartridge, chip, or card with fluidic structures (e.g., channels, chambers, voids, etc.) having microfluidic dimensions, e.g., at least one internal cross-sectional dimension that is less than approximately 500 μm to 1000 μm and typically between approximately 0.1 μm and approximately 500 μm. These fluidic structures may include chambers, valves, vents, vias, pumps, inlets, nipples, and detectors and sensors, for example. The microfluidic flow regime is characterized by Poiseuille or “laminar” flow. (See, e.g., Staben et al. 2005. Particle transport in Poiseuille flow in narrow channels. Intl J Multiphase Flow 31:529-47, and references cited therein).

Microfluidic devices may be fabricated from various materials using techniques such as laser stenciling, embossing, stamping, injection molding, masking, etching, and three-dimensional soft lithography. Laminated microfluidic devices are further fabricated with adhesive interlayers or by thermal adhesiveless bonding techniques, such as by pressure treatment of oriented polypropylene. The microarchitecture of laminated and molded microfluidic devices can differ. In certain embodiments, the microfluidic devices of the present technology are designed to interact or “dock” with a host instrument that provides a control interface and optional temperature and magnetic interfaces. The card, however, generally contains all biological reagents needed to perform the assay and requires only application of a sample or samples. These cards are generally disposable, single-use, and are generally manufactured with sanitary features to minimize the risks of exposure to biohazardous material during use and upon disposal.

As used herein, the term “sample” includes, but is not limited to, biological samples such as, e.g., tissue samples such as a sample of a soft biological tissue. In exemplary embodiments, the soft biological tissue is a neural tissue such as a piece of brain tissue or an entire brain. In some embodiments, samples are placed directly in the device; in other embodiments, samples are processed prior to analysis (e.g., by treating with enzymes).

As used herein, the term “reagent” refers broadly to any chemical or biochemical agent used in a reaction, including enzymes. A reagent can include a single agent which itself can be monitored (e.g., a substance that is monitored as it is heated) or a mixture of two or more agents. A reagent may be living (e.g., a cell) or non-living. Exemplary reagents utilized in embodiments of the present disclosure include, but are not limited to, enzymes for partially degrading the extracellular matrix, culture medium (e.g., buffered salt solutions, insulin, and fetal bovine serum), and antibiotics. Not all reagents are reactants.

As used herein the term “detergent” refers to anionic, cationic, zwitterionic, and nonionic surfactants.

As used herein, the term “microfluidic channel” or “microchannel” refers to a fluid channel having a variable length and one dimension in cross-section less than 500 to 1000 μm.

As used herein, a “check valve” is a one-way valve.

As used herein, the term “via” refers to a step in a microfluidic channel that provides a fluid pathway from one substrate layer to another substrate layer above or below, characteristic of laminated devices built from layers.

As used herein, a “detector” or “sensor” refers to an apparatus for detecting a signal associated with the endpoint of an assay (e.g., to detect dissociation of tissue) or for detecting a signal associated with monitoring an assay in real time (e.g., for monitoring dissociation of tissue). In some embodiments, a detector or a sensor includes a detection channel. In some embodiments, a detector or sensor includes but is not limited to, e.g., a spectrophotometer, fluorometer, luminometer, photomultiplier tube, photodiode, nephlometer, photon counter, voltmeter, ammeter, pH meter, capacitative sensor, radio-frequency transmitter, magnetoresistometer, or Hall-effect device. Magnetic particles, beads, and microspheres having color or impregnated with color or having a higher diffraction index are used in some embodiments to facilitate visual or machine-enhanced detection of an assay endpoint. Magnifying lenses, optical filters, colored fluids, and labeling are used in some embodiments to improve detection and interpretation of assay results. A detector or a sensor may detect a signal produced by a “label” or “tag” such as, but not limited to, dyes such as chromophores and fluorophores, radio frequency tags, plasmon resonance, spintronic, radiolabel, Raman scattering, chemiluminescence, inductive moments, fluorescence quenching, or fluorescent proteins synthesized by genetically engineered cells. Detection systems are optionally qualitative, quantitative, or semi-quantitative. Visual or optical detection is preferred for its simplicity; however, a detector or sensor can comprise visual detection, machine detection, manual detection, or automated detection.

As used herein, a “heating and cooling” includes convective and conductive heating and cooling elements such as electroresistors, hot air, lasers, infrared radiation, Joule heating, thermoelectric or Peltier devices, heat pumps, endothermic reactants, and the like, generally in conjunction with a heat sink for dissipating heat. Heating also includes heating by the motion of magnetic beads driven by a high frequency magnetic field.

As used herein, the terms “dissociated”, “dissociated state”, “dissociation”, etc. refer to a state in which an input sample (e.g., a tissue, organ, etc.) comprising associated cells has been processed into an output sample comprising individual cells such that no clumps or clusters of two or more cells are present. In some embodiments, dissociation is expressed in relative terms indicating a fraction or a percentage of a collection of cells that are in the dissociated state. In some embodiments, the fraction or percentage of dissociated cells in an output sample is provided relative to the number of cells in the input sample. In some embodiments, the fraction or percentage of dissociated cells in an output sample is provided relative to the total number of cells in all states (e.g., associated, dissociate, other) in the output sample. An output sample may comprise only dissociated cells or it may comprise some cells that are dissociated and some cells that are not dissociated (e.g., that remain “associated” in small clusters).

As used herein, the term “viable” and “viability” (e.g., for neurons that are cultured after dissociation) refers to a level or state of survival of cells (e.g., neurons). In some embodiments, three phases of viability are assayed. (1) Survival—cell is still alive (by biochemical criteria) immediately after dissociation and plating in a culture well. (2) Extension, from the cell body, of neurite(s) that undergo branching. This takes place over several days to weeks (depending on the species; fly neurons are much faster than rodent neurons). (3) Attainment of other features of differentiation, including the shape of the neurite arbor and expression of proteins that confer neuron-type-specific chemical phenotype, including the neurotransmitter it synthesizes and the neurotransmitter receptors on its surface; adhesion molecules that promote contact with other neuron cell types, etc. Depending on the neuron type, phase (3) could occur in parallel with phase (2) or somewhat delayed. In some embodiments, at least 20% (e.g., at least 30%, 40%, 50%, 60%, at least 75%, at least 80%, at least 90%, at least 95%, at least 99%, etc.) of cells are viable (e.g., as assayed using the phases of viability described above) after dissociation.

### Description

Although the disclosure herein refers to certain illustrated embodiments, it is to be understood that these embodiments are presented by way of example and not by way of limitation.

### Fabrication of Microdevices

The technology relates to the use of microdevices for tissue dissociation. In some embodiments, the cartridge is generally fabricated using one or more of a variety of methods and materials suitable for microfabrication techniques. For example, in some embodiments the body of the device comprises a number of planar members that are individually injection molded parts fabricated from a variety of polymeric materials, or that are silicon, glass, or the like. In the case of crystalline substrates like silica, glass, or silicon, methods for etching, milling, drilling, etc. are used to produce wells and depressions that compose the various reaction chambers and fluid channels within the cartridge. Microfabrication techniques, such as those regularly used in the semiconductor and microelectronics industries, are particularly suited to these materials and methods. These techniques include, e.g., electrodeposition, low-pressure chemical vapor deposition, photolithography (e.g., soft photolithography), etching, laser drilling, and the like. Where these methods are used, it will generally be desirable to fabricate the planar members of the device from materials similar to those used in the semiconductor industry, e.g., silica, silicon, or gallium arsenide substrates. U.S. Pat. No. 5,252,294, incorporated herein by reference in its entirety for all purposes, reports the fabrication of a silicon based multi-well apparatus for sample handling in biotechnology applications.

In some embodiments, photolithographic methods of etching substrates are particularly well suited for the microfabrication of these microfluidic cartridges. For example, the first sheet of a substrate may be overlaid with a photoresist. An UV-radiation source may then be shined through a photolithographic mask to expose the photoresist in a pattern that reflects the pattern of chambers and/or channels on the surface of the sheet. After removing the exposed photoresist, the exposed substrate may be etched to produce the desired wells and channels. Generally preferred photoresists include those used extensively in the semiconductor industry. Such materials include polymethyl methacrylate (PMMA) and its derivatives, and electron beam resists such as poly(olefin sulfones) and the like (more fully discussed in, e.g., Ghandi, “VLSI Fabrication Principles,” Wiley (1983) Chapter 10, incorporated herein by reference in its entirety for all purposes).

Although primarily described in terms of producing a fully integrated body of the microfluidic cartridge, the methods provided are used in some embodiments to fabricate individual discrete components of the microfluidic cartridge which are later assembled into the body of the microfluidic cartridge.

In additional embodiments, the microfluidic cartridge comprises a combination of materials and manufacturing techniques. In some embodiments, the microfluidic cartridge includes some parts of injection-molded plastics, and the like, while other portions of the microfluidic cartridge comprise etched silica or silicon planar members, and the like. In some embodiments, the microfluidic cartridge includes some parts formed by photolithography, and the like, while other portions of the microfluidic cartridge comprise glass, etched silica, or silicon planar members, and the like. For example, in some embodiments, injection molding techniques are used to form a number of discrete cavities in a planar surface that define the various components, modules, and/or reaction chambers, whereas additional components, e.g., fluid channels, arrays, etc, are fabricated on a planar glass, silica or silicon chip or substrate. Lamination of one set of parts to the other then results in the formation of the various reaction chambers, which are interconnected by the appropriate fluid channels.

In some embodiments, the microfluidic cartridge is made from at least one injection molded, press-molded, or machined polymeric part that has one or more wells or depressions manufactured into its surface to define several of the walls of the reaction chamber or chambers. Examples of suitable polymers for injection molding or machining include, e.g., polycarbonate, polystyrene, polypropylene, polyethylene acrylic, and commercial polymers such as Kapton®, Valox®, Teflon®, ABS (acrylonitrile-butadiene-styrene), Delrin®, and the like. A second part that is similarly planar in shape is mated to the surface of the polymeric part to define the remaining wall of the reaction chamber(s).

In some embodiments, the microfluidic devices are prepared using multilayer soft lithography techniques. For example, in some embodiments of the technology relates, microfluidic devices are prepared as multilayer polydimethylsiloxane (PDMS) (e.g., Sylgard 184) devices (e.g., on a solid substrate, e.g., on glass) using multilayer soft lithographic (MSL) techniques. See, e.g., Unger et al (2000) Science 288: 113-116 and International Patent Application WO2001001025, each incorporated herein by reference in its entirety.

Accordingly, during the development of the technology provided herein, microdevices were fabricated and tested. Microdevices were fabricated having various configurations, e.g., by changing the size and spacing of the orifices along the dissociation channel. In some exemplary embodiments, PDMS and soft lithographic techniques were utilized to fabricate the microchannels as it is inexpensive, bio-compatible, optically clear and widely used for manipulation of biological species.

For example, in some embodiments a silicon wafer and a “mask” of the microchannel design are used to generate a mold for the fluidic channels by photolithography. The silicon wafer is cleaned, dehydrated, and coated with hexamethyldisilazane (HMDS) for improved adhesion. Photoresist (e.g., Micro-Chem Corp. SU-8) is spun onto the wafer surface to form a uniform film of approximately 250 μm to 500 μm thickness. After soft baking, the photoresist film is exposed to UV light using a mask aligner and developed to transfer the microchannel patterns to the photoresist film to create the mold for the microchannels with the desired shapes and dimensions. PDMS mixture is prepared and poured over the SU-8 mold to cast the microchannels. The PDMS is cured overnight at room temperature and the microchannels are peeled off the mold. A single mold may contain the designs for a number of different microdevices, either of the same or different configurations. Also, the mold can be reused repeatedly to fabricate numerous devices.

Microfluidic device fabrication is completed by bonding the PDMS microchannel to a flat PDMS substrate of approximately 3 mm thickness following oxygen-plasma treatment of both bonding surfaces. Finally, adaptors are attached to the device inlet/outlet holes connecting the microdevice to the external fluid-handling system. The surface of cured PDMS is highly hydrophobic, thus allowing easy tissue manipulation without sticking to the channel walls. PDMS devices can be sterilized by autoclaving, UV radiation, or surface cleaning with ethanol. Because of the low cost associated with these techniques, in some embodiments the fabricated device is disposable and thus eliminates cleaning time and the risk of cross-contamination between experiments.

Using this technology, many (e.g., 10 to 100, e.g., 50 to 150) microdevices can be fabricated and tested, thus providing a rapid method to prototype and test various configurations of channels, orifices, and other features of the microdevice. Further, the fabrication technology provides for systematic design, fabrication, and testing of microdevices to test tissue dissociation and flow parameters.

The devices described herein are suitable for use with a variety of cell and tissue types. Examples include, but are not limited to, organ (e.g., brain, spinal cord, heart, lung, etc., tissue), regionally dissected parts of an organ (e.g., hippocampus, cerebellum, parietal cortex, etc., of the brain), or any glandular tissue, skin, and the like. Tissue derived from any animal or organism may be dissociated using the systems and methods described herein.

The devices and systems described herein find use in a variety of research, screening, and clinical applications. Applications include, but are not limited to, obtaining live cells for drug screening, therapeutic, or research uses. For example, in some embodiments, devices and systems described herein are used to obtain live cells, which are cultured and used to assay the effect of a test compound (e.g., drug or candidate drug) on one or more functions, activities or viability of the cells.

### EXAMPLES

### Example 1—Microsystem Assembly

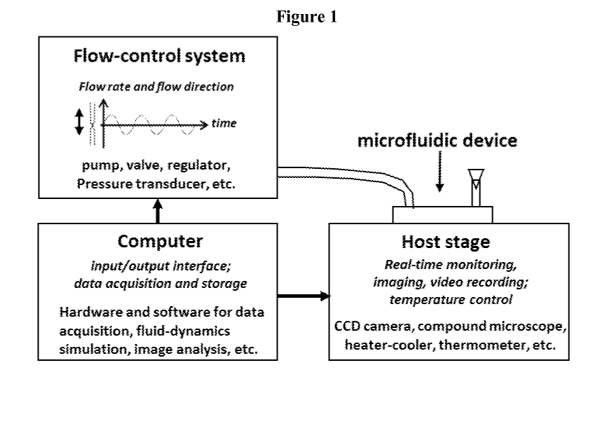

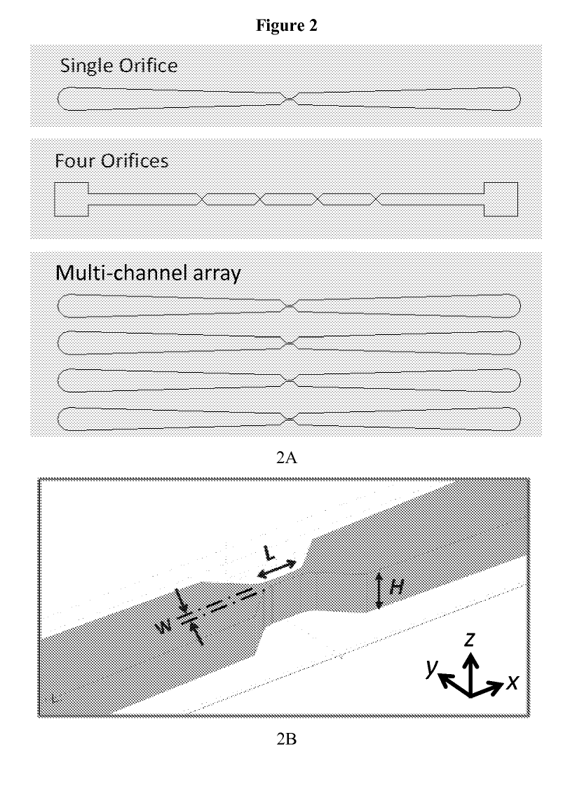

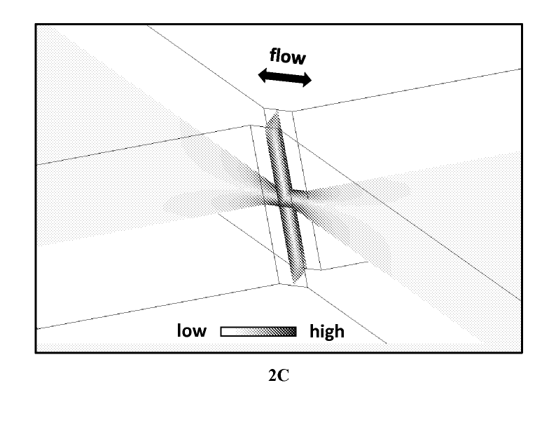

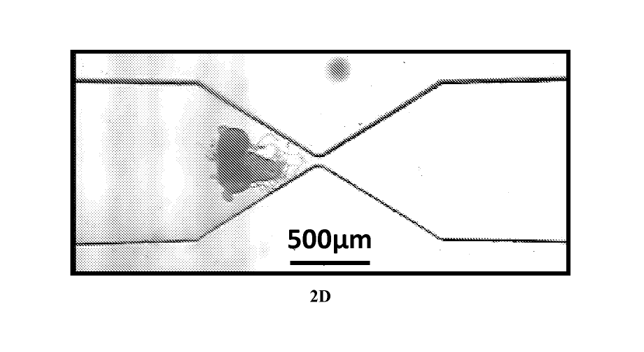

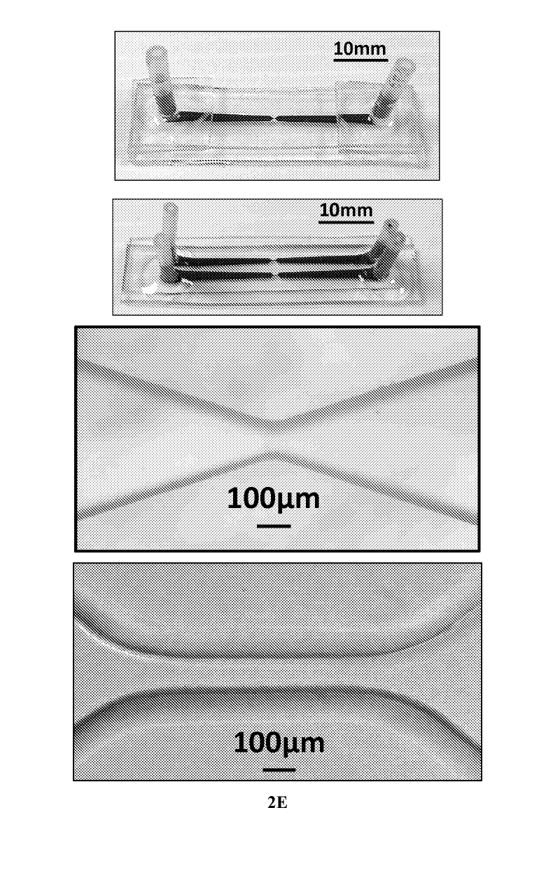

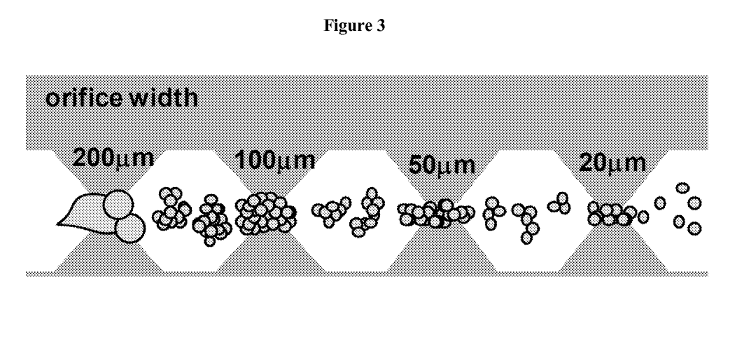

An exemplary embodiment of the system as described above is shown in FIG. 1. In some embodiments, the system comprises a programmable pump (e.g., a syringe pump), valves, and a pressure transducer, which regulate and manipulate the flow according to the flow parameters, e.g., regulating flow direction and magnitude, to provide a controlled flow field within the microfluidic device. In some embodiments, the pump generates continuous flow; in some embodiments the pump generates oscillatory flow according to flow parameters such as, e.g., flow rate, oscillation frequency, and number of cycles.

In an exemplary embodiment, a transparent fixture (e.g., one or more acrylic plates) holds the microdevice and its tubing system in place to provide a system herein referred to by the term “packaged microdevice”.

In some embodiments, the entire microfluidic system (including tubing, syringes, and adapters) is sterilized, e.g., by autoclaving, exposure to ozone, exposure to ionizing radiation, washing with ethanol, etc., to prevent microbial contamination (e.g., prior to tissue sample loading).

During the development of embodiments of the technology provided herein, microsystems were designed and fabricated according to various configurations and tested according to various exemplary methods. For example, some tests used tissue samples that were acquired by microdissection of the developing central nervous system (herein called “brain tissue”) of a fruit fly at a particular developmental stage. Then, the tissue was treated with an enzyme, e.g., a standardized blend of collagenase and a neutral protease (e.g., dispase), e.g., as sold commercially under the trade name “Liberase” by Roche Applied Science (Gill et al., Transplantation Proceedings, 27(6): 3276-3277, 1995). In some embodiments, enzyme treatment is for 1 hour at ambient (“room”) temperature. Enzyme type, enzyme concentration, and incubation time are varied to configure the device for processing different sizes and types of biological tissues.

Then, in exemplary embodiments, enzyme-treated tissue is drawn (e.g., with its culture medium) into a pipette tip and loaded into the device outlet, from which the sample-containing medium is driven into the microchannel (e.g., in some embodiments by manipulating the pressure difference between the channel inlet and outlet using the valves, e.g., in some embodiments, under visual guidance by video microscopy).

The syringe pump, or a pumping system of any type, is programmed according to a set of flow parameters to deliver a periodic oscillating flow, where the flow rate, periodic frequency, and number of cycles are adjustable, e.g., adjustable by a user (e.g., a user manipulating a user interface) during processing of the tissue or adjustable by software, e.g., adjustable by software receiving real-time data describing the tissue dissociation (e.g., data from a sensor such as an image sensor (e.g., a video sensor (e.g., a charge coupled device (CCD)).

In some embodiments, systemic study of the dissociation of various tissue types and amounts establishes the various configurable features of the system, e.g., enzyme treatment (e.g., enzyme type, enzyme concentration, incubation time, and incubation temperature), flow rate, oscillation frequency, and number of cycles, which are associated with dissociating the cells of the various tissue types and amounts. In some embodiments, the systematic study produces an enzyme treatment and/or a set of flow parameters that generates optimal shear stress for dissociation of the tissue amount and type.

In experiments conducted during the development of the technology provided herein, data were collected indicating that dissociation is rapid following the start of operation, and readily observable via the real-time video component as clouds of cells and cell clumps are released from the brain tissue as it is driven back and forth through the orifice of the microfluidic device. Data further indicated that dissociated cells after the first phase of dissociation were not readily observable at the same optical magnification, while clumps of cells, if there are any, could be seen. Accordingly, in experiments conducted during the development of the technology provided herein, after no clumps of cells could be observed except for leftover neuropil, the cell suspension was collected at the exit of the microdevice, and prepared accordingly for in vitro culture for several days in a standard culture well to assess its health, viability, and state of dissociation.

In some embodiments, the technology provides an efficient technique for the dissociation of tissue. For example, experiments conducted during the development of embodiments of the technology indicated that the residence time of the brain tissue and its dissociated components within the microchannel, e.g., from sample loading to cell collection, is approximately 3 minutes to approximately 5 minutes.

During the development of the technology provided herein, measured data were collected to compare the flow field in response to the system inputs in the developed microchannel to predictions based on numerical simulations. Accordingly, the combined effects of enzymatic treatment and flow-shear cyclic load are configurable to provide chemical and mechanical loading conditions for efficient tissue dissociation and cell viability.

In some embodiments, the system comprises a microscope (e.g., a stereomicroscope, or compound microscope with long-working-distance objectives, and image sensor), a computer (e.g., a microprocessor), and a pump (e.g., a programmable pump, e.g., a programmable syringe pump). In some embodiments, the system comprises one or more flow valves and/or a pressure transducer, e.g., to provide quantitative control of the flow field. In some embodiments, the connections between the “packaged microdevice” and the external fluid-handling system provide for reliable and efficient tissue loading and cell collection. Further, in some embodiments the system is automated, e.g., using software to control operation of the system components to start the pump, stop the pump, control the flow rate, control the oscillation frequency, and control the number of cycles.

### Example 2—Configuration of Microfluidic Systems

During the development of some embodiments of the technology provided herein, the configuration of the microdevice design and operation were evaluated by flow field simulations performed in parallel with empirical dissociation experiments and cell culture analyses, e.g., to determine flow parameters and enzyme treatments for various tissue types and sizes. For example, experiments were conducted to collect data from numerical simulations of a transient three-dimensional flow field in the absence of tissue. These data provided an understanding of the flow field and the associated shear-stress level in response to the imposed boundary and initial conditions. In addition, simulations of the flow field in the presence of tissue in the microdevice provide data relevant to operational parameters such as channel dimensions, orifice dimensions, and operation conditions (e.g., flow parameters such as, e.g., flow rate, oscillation period, cycle number). The numerically computed and experimentally estimated stress levels are compared and evaluated with respect to the tissue dissociation provided by the system operating according to the operational parameters.

### Example 3—Cell Yield and Viability During In Vitro Culture

Further, the health and viability of cells produced by the technology are evaluated by collecting data from tests performed on dissociated cells recovered from the device. In particular, after removal from the device, the cells are washed, gently centrifuged, re-suspended in fresh culture medium, and plated in standard dishes with substrate-coated glass wells with gridded bottoms for ease of tracking. For each protocol and design variant, comparison is made to conventional cultures prepared by manual trituration (Higgins and Banker, Primary dissociated cell cultures. In Culturing Nerve Cells, G. Banker and K. Goslin, Editors. 1998, The MIT Press: Cambridge, Mass. p. 37-78). Several outcome measures are evaluated, e.g., by phase-contrast and/or fluorescence microscopy (e.g., at 400-600×): dissociation of cells, recovery of cells; survival of cells over 4 days following dissociation; and, for neurons, extent of neurite outgrowth as a manifestation of neuronal health and capacity to differentiate. In some experiments, neurite-arbor analysis is performed after immunofluorescent labeling using NeuronMetrics™ software (Narro, et al., Brain Res, 1138: 57-75, 2007).

Assessment of viability is performed using, e.g., a LIVE/DEAD assay (Molecular Probes), in which live cells are green fluorescent and dead and/or dying cells are red-fluorescent.

For morphometric analyses of certain cells (e.g., neurons), neurons are evaluated with respect to their recovery and neurite outgrowth parameters compared to those measures following conventional manual dissociation (Kraft et al., J. Neurosci., 18: 8886-8899, 1998; Kraft et al., J Neurosci, 26(34): 8734-47, 2006). The neurite-arbor size and shape measures include length, branch count, territory, higher-order branch density, axon:dendrite length ratio. For identifiable classes of neurons, e.g., y mushroom body neurons from the Drosophila brain, data are collected to evaluate plasticity based on increased neurite outgrowth in response to the steroid molting hormone 20-hydroxyecdysone. As an additional test of biological fidelity, mutant neurons dissociated in the microfluidic device retain their phenotypic neurite arbor characteristics when cultured in vitro. Accordingly, data collected produce configuration and operational parameters for the technology that maximize cell yield, cell survival, and preservation of wild-type or mutant characteristics, without the fatigue and potential inconsistencies associated with manual trituration, indicating increased efficiency of cell culture preparation.

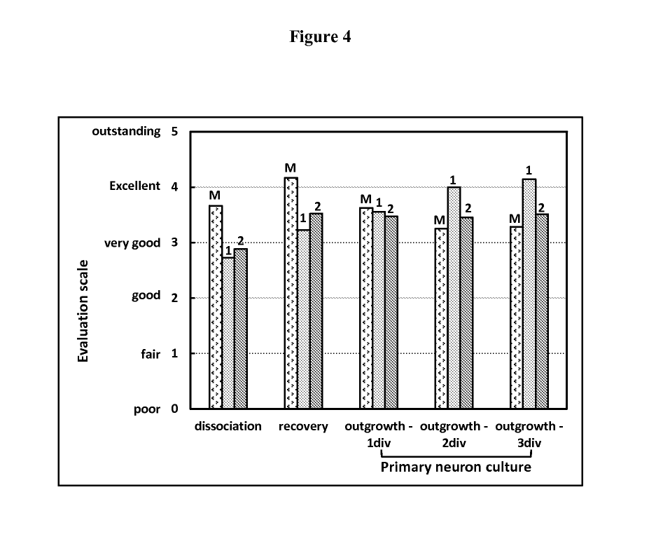

As shown in FIG. 4, data collected for dissociation, recovery, outgrowth (1 day in vitro, 1 div), outgrowth (2 days in vitro, 2 div), and outgrowth (3 days in vitro, 3 div) for tissue dissociated using the system described herein, independently by two operators (“1” and “2”) and compared with existing methods (“M”), indicates that dissociation using the microsystem (“1” and “2”) delivers neurons capable of neurite outgrowth, comparable to those cultures prepared by manual trituration (“M”).

### Example 4—Microfluidic Devices

Device Fabrication and Experiment Preparations:

Microdevices were made using PDMS, a transparent polymer, and assembled with inlet/outlet tubing for connection to an external flow control system. The devices were cleaned with 70% ethanol and, subsequently, treated under UV light prior to experiments. Procedures were followed to ensure sterile conditions within the fluidic system.

Tissue Samples:

The microdevices were primarily designed based on the size of a Drosophila larval central nervous system (CNS); thus, an intact CNS tissue could be loaded and dissociated in the devices. Fetal (E18) rat hippocampal tissue and neonatal (P2) rat heart tissue were cut into individual samples about 1-2 mm in size for experiments. The enzyme treatment procedures, either established by individual labs for manual dissociation or available protocols for commercial products, were followed without major changes (including concentration, temperature, time and media). Upon completion of the enzyme treatment, tissue samples were immediately transferred to fresh culture media and loaded into the device through its outlet.

Dimensions of the Constriction

Different types of tissue samples can be dissociated using microdevices with the same configuration (e.g., geometry and dimensions), as long as the sample size is within the designed operation range. Repeatability of dissociation results was demonstrated using a single device configuration for three types of soft tissue samples. In addition, the elasticity of PDMS as the device structure material is advantageous. The device constriction dimensions are finely adjusted (reduced) to accommodate specific requirements due to variations in tissue type and sample size. It also allows dissociation in a two-step process: dissociate the sample for time t₁ through the constriction with cross-section A₁, and further dissociate the sample for time t₂ through the constriction with cross-section A₂, where A₂<A₁. This two-step process, though not necessary, allows more efficient and complete dissociation when the device has a single orifice rather than a series with progressively decreasing widths.

Flow Control

The flowrate and frequency are important parameters to achieve adequate dissociation since they dictate, dependently, the stress loads on the samples, which determine the dissociation results in terms of degree of dissociation, yield, and viability. For the devices and samples described here, the dissociation flowrate range was 50-120 μl/sec, while the frequency range was 3-5 Hz.

Although the same device configuration can be used for dissociation of various types, the stress loads are adjusted depending on the type of tissue and cells. Among the three tissue types described here, the two brain samples—Drosophila larval CNS and rat E18 hippocampal tissues—are more sensitive to the magnitude of the applied stress loads. Smaller stress loads over longer period of time with more cycles yield improved and repeatable dissociation results.

For rat P2 heart tissue samples, higher stress loads were acceptable. The tissue broke down into small clusters of cells within 30 sec into the dissociation process with many individual cells. Within 1 minute, only individual cells were observed inside the channel with few small cell clusters. Dissociation for 2 more minutes led to 100% dissociation with no visible cell clusters. Good and repeatable results in terms of dissociation degree, yield and cell viability were obtained.

Cell Collection

Upon completion of the dissociation process, cells were collected into 1.5 ml centrifugal tubes in preparation for culture. The volume of the collected cell suspension depends on the sample size. For each of the Drosophila CNS samples, 500 μl was collected and 1.5 ml was collected for each of the rat brain and heart tissue samples.

To check the dissociation degree and yield using the microdevices, the number of cells collected after dissociation were counted using a hemocytometer. Rat hippocampal tissue was used for this test since the tissue is a commercial product from BrainBits, LLC, with a guarantee to yield 1.0 million viable cells from a pair of rat hippocampal tissue. Results indicated that 2.2×10⁵ and 2.3×10⁵ cells were obtained for two randomly selected samples, respectively; each sample is ⅛ of the pair of rat E18 hippocampi. A rough estimate shows that more than 1.5 million cells were obtained from the pair of the tissue, indicating good dissociation degree and yield. Cell viability was investigated based on the results of neuron survival and outgrowth in subsequent cell culture. Table 1 summarizes the overall parameters of the experiments.

TABLE 1
Tissue-Dissociation Parameters in Microfluidic Devices

| | | Sample | | | | | | |
|---|---|---|---|---|---|---|---|---|
| | | maximum | | | | | | |
| | | dimension | Cell size | Flowrate | Frequency | Time | | Manual |
| Tissue type | Sample | (mm) | (μm) | (μl/sec) | (Hz) | (min) | Cycles | dissociation |
| Drosophila brain tissue | whole CNS | 0.5 | 5-10 | 50 | 5 | 5 | 1400 | Y |
| (wild and mutant) | | | | | | | | |
| Rat E18 hippocampal tissue | 8 pieces | 1-2 | 10-20 | 50 | 4 | 5 | 1400 | N/A |
| | (one pair) | | | 100 | 3.3 | 3 | 600 | |
| Rat P2 heart tissue | 64 pieces | 1-2 | 10-20 | 100 | 3.3 | 3 | 600 | N/A |
| | (one rat pup heart) | | | | | | | |

### Example 5—Micro-Scale Automated Tissue Dissociation for Primary Cell Culture

This example describes analysis of devices for micro-scale automated tissue dissociation and primary cell culture.

Microdevice design & fabrication as well as optimization of the operating conditions for the dissociation of wild-type & mutant Drosophila (fruit fly) CNS tissue samples from developing animals (larvae) was performed. Four device configurations were tested. A total of 45 tissue-dissociation experiments, including 26 wild-type and 19 mutant samples were performed. Cells from each CNS were recovered from the device and plated for in vitro culture.

Assessments of extent of dissociation and cell yield were done at the time of plating; cell survival and neurite outgrowth were assessed daily, and about half of the cultures were immunostained after 3 days for quantification of neurite outgrowth. As controls for all of the biological reagents used for culturing the cells after dissociation, nine manual dissociations, each in parallel to several done in the microdevices, were performed.

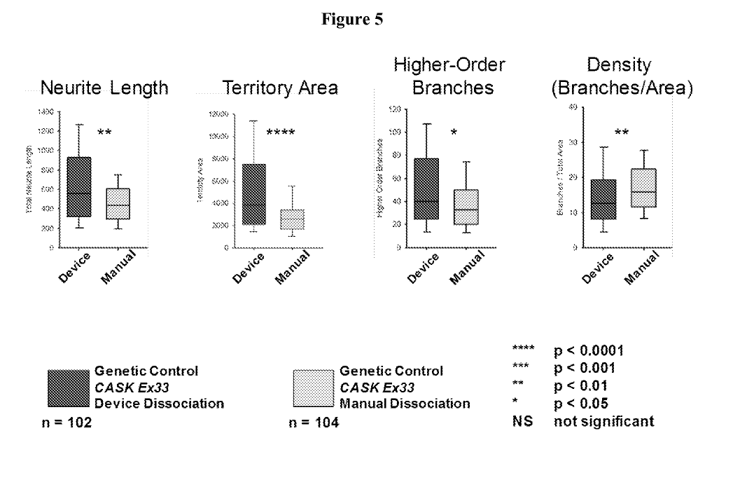

Neurite outgrowth from neurons dissociated in the device was remarkably good by all quantitative parameters. Microbial contamination was not a significant problem. An optimized device configuration and a set of flow control parameters were obtained that yield reliable and consistent dissociation results for Drosophila neurons (See FIGS. 5, 6A, 6B, 7A, 7B).

FIG. 5 shows that genetically normal neurons extend larger neurite arbors when they are dissociated in the microfluidic device, compared with a parallel manual dissociation. The left panel of each graph shows neurite length of neurons dissociated using the device and the right panel shows neurite length of neurons dissociated manually.

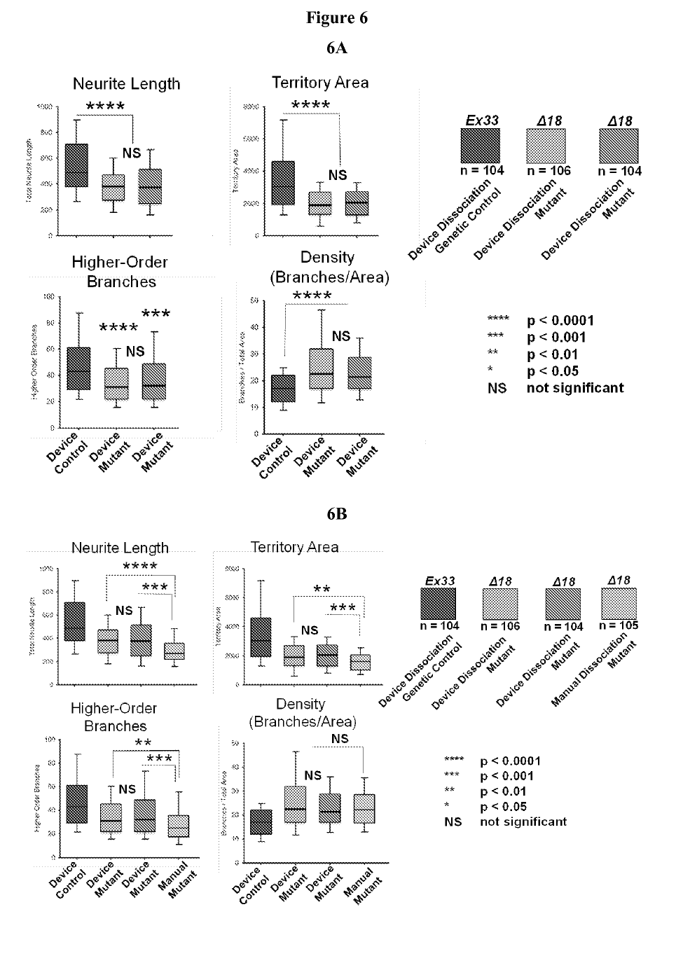

FIG. 6A shows that when mutant and control CNS are dissociated in the device, the CASK-mutant neurons extend smaller, denser neurite arbors, compared with genetic-control neurons. This replicates the CASK-mutant phenotype previously shown in manual cultures. The left panel in each box-plot represents genetic-control (CASK Ex33) neurons, the center box-plot represents CASK-mutant (Δ18) neurons, and the right box-plot represents a duplicate sample of CASK-mutant (Δ18) neurons. This figure also shows that independent duplicate preps of mutant neurons dissociated in the device are very consistent.

FIG. 6B shows that CASK-mutant neurons cultured after dissociation in the device are larger than those from a parallel manual culture. From left to right, each box-plot represents device-dissociated control (CASK Ex33) neurons, device-dissociated mutant (CASK Δ18) neurons, a duplicate sample of device-dissociated mutant (CASK Δ18) neurons, and manual-dissociated mutant (CASK Δ18) neurons.

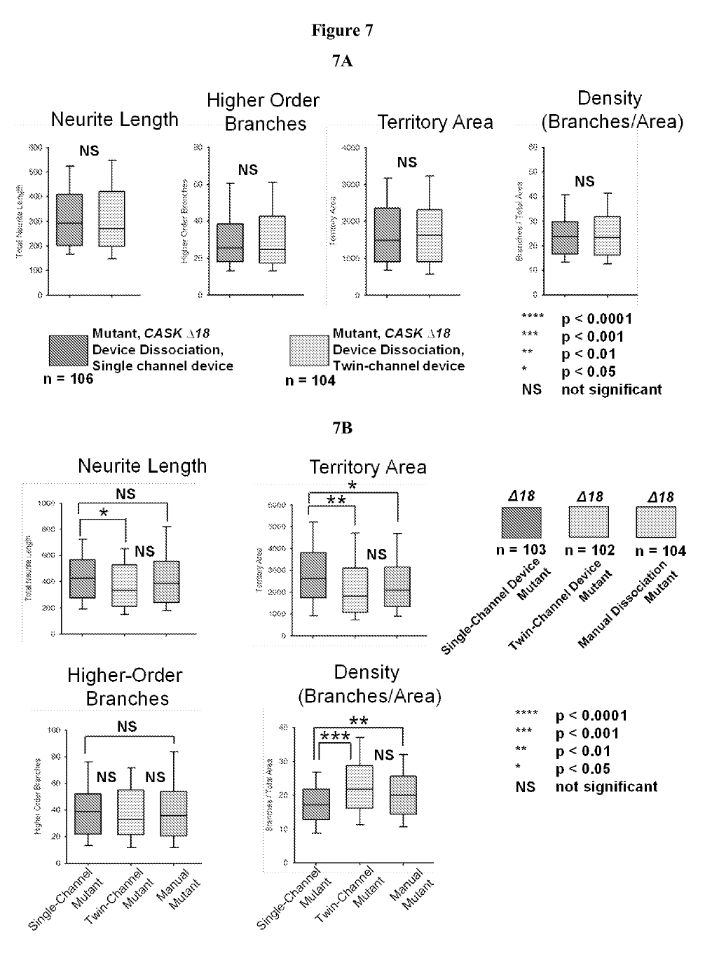

FIG. 7A shows that neurons cultured after dissociation in twin- and single-channel devices extended arbors of very similar size. The left panel represents neurons dissociated using single-channel devices and the right panel represents neurons dissociated using twin-channel devices. FIG. 7B shows that, in an independent experiment, neurons cultured from twin- and single-channel devices grew arbors of similar size, both to each other, and to those dissociated manually. The left panel represents neurons dissociated using single-channel devices, the center panel represents neurons dissociated using twin-channel devices, and the right panel represents neurons dissociated manually.

Experiments were performed to dissociate mammalian brain tissue, mostly rat hippocampus from E18 (embryonic day 18), into viable neurons. The tissue samples were purchased from BrainBits with a pair of hippocampi in each overnight shipment. Each hippocampus was further cut into 4 pieces with maximum dimension ˜1 mm. Experiments were performed to dissociate a total of 9 rat hippocampi (36 pieces) and 1 mouse hippocampus. Reagents that promote the survival of neurons, but not glia were used. Qualitative assessments of the cultured neurons were performed; the cultures were not assayed long enough to determine details of neuronal differentiation. Note that Drosophila neurons differentiate much more quickly in vitro than do mammalian neurons, 3 days vs. 3 weeks.

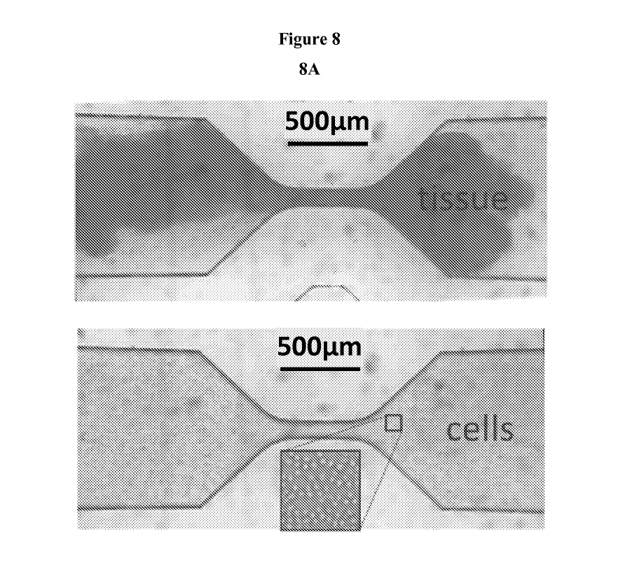

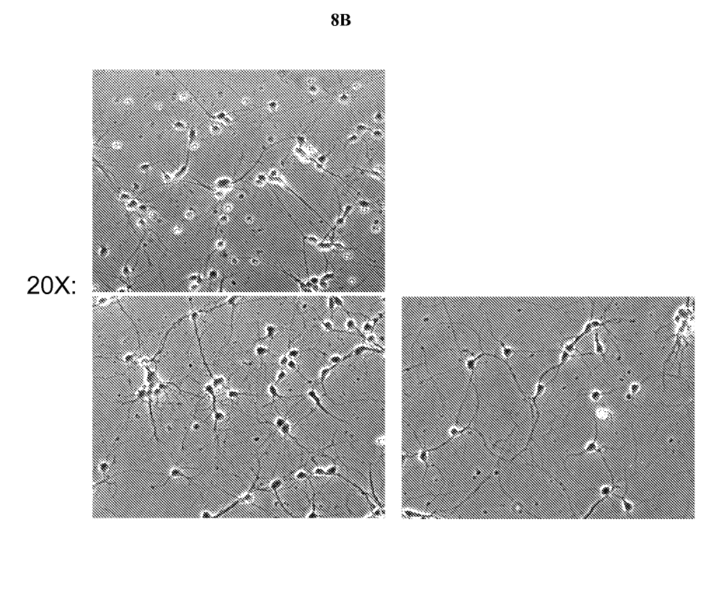

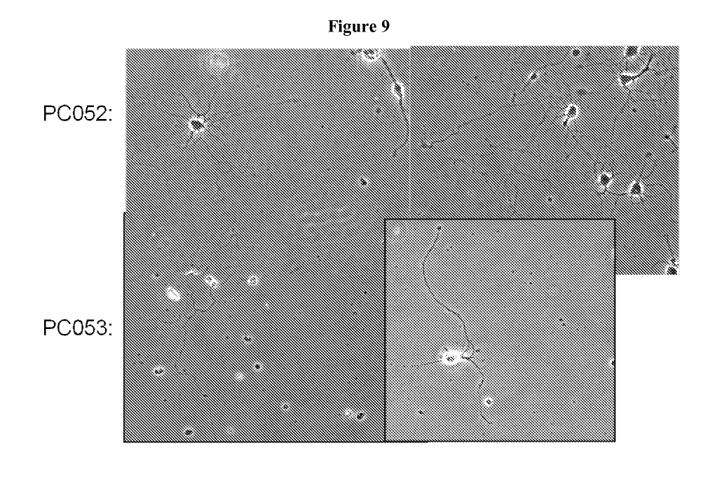

Flow-control parameters and dissociation-process steps were adjusted to obtain good dissociation results in terms of cell yield, viability, and neurite outgrowth. In order to obtain single neurons, as opposed to small clusters (2-5 cells), a two-step approach that narrowed the constriction and the height of the channel midway through the dissociation was used (See FIGS. 8A, 8B, 9).

The top panel of FIG. 8A shows a piece of intact rat E18 hippocampus tissue loaded into the device and driven by the pump through the orifice at the channel constriction. The bottom panel shows cells collected immediately after dissociation. FIG. 8B shows neuronal cell culture and outgrowth of dissociated rat hippocampus neuronal cells. The figure shows that the neurite outgrowth was robust and the cells were healthy. FIG. 9 shows neurons cultured using a two-step protocol. With the two-step protocol, brain-tissue dissociation within the microdevice is complete, yielding single neurons. When plated at low density, the isolated neurons grow complex arbors in vitro.

Experiments were performed to dissociate rat heart tissue, as an example of non-brain soft tissue, to determine if the microdevice method would allow preparation of cardiomyocyte cultures. Fresh heart tissue from 2-day-old (P2) rat pups was obtained. Tissue comprising approximately one cardiac ventricle was cut into 16 roughly cuboid samples ranging in size between 1 and 2 mm. Dissociation experiments were performed on 32 rat heart tissue samples. The procedure standard procedure for dissociation yields a combination of red blood cells, fibroblasts, and well-isolated cardiomyocytes. A two-step plating method can be done to remove the fibroblasts.

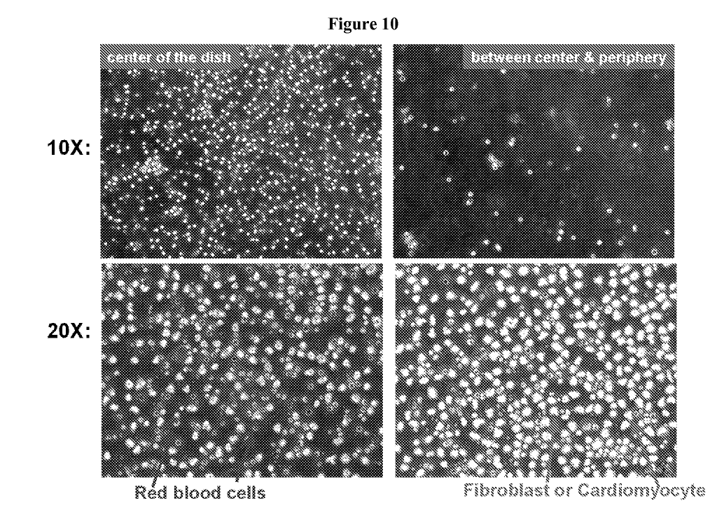

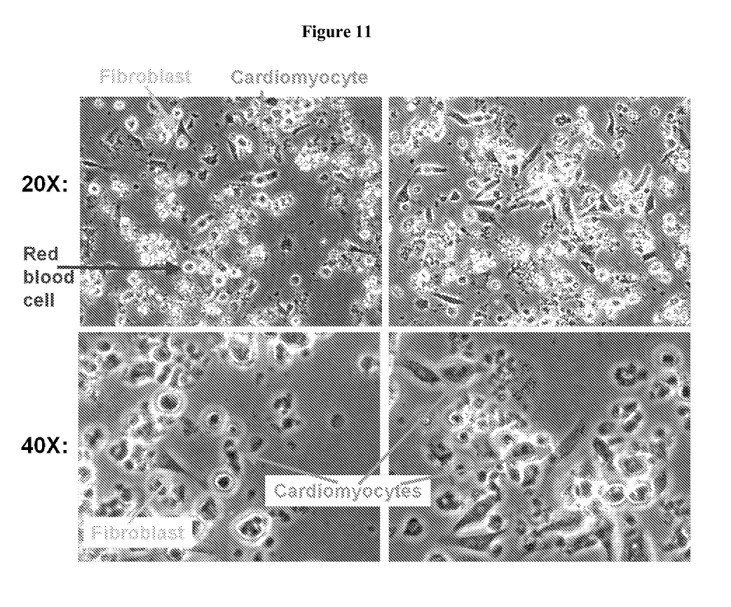

A protocol was developed that includes enzyme-treatment time, flow-control parameters and dissociation-process steps to obtain viable cells, of all three types, with high yield. Immediately after plating, the cardiomyocytes and fibroblasts could not be distinguished, but over the following day in culture, they begin to differentiate and each type developed a distinctive appearance (FIGS. 10 and 11).

FIG. 10 shows images of heart muscle cell culture at 0 div. As shown in FIG. 11, by 1 div, fibroblasts and cardiomyocytes had distinct appearances.

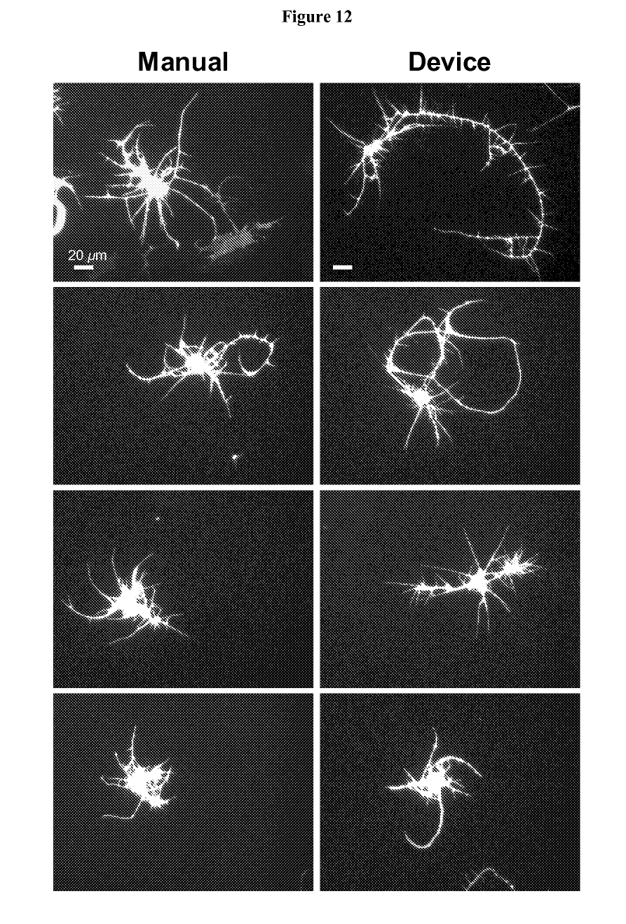

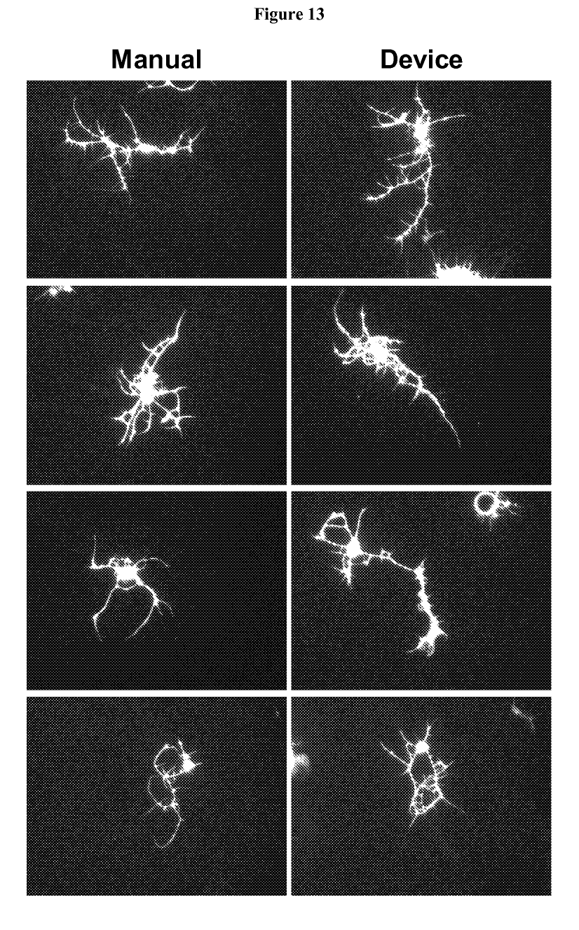

FIGS. 12 and 13 show representative Drosophila neurons cultured after dissociation by manual or microfluidic device methods: Brain tissue from genetic control animals (FIG. 12) and Brain tissue from CASK-mutant animals (FIG. 13).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A system for dissociating an input tissue sample into an output sample comprising viable cells, the system comprising:
a) a microfluidic device comprising an orifice configured to induce a stress field on a tissue sample; and
b) a programmable pump configured to transport the tissue sample cyclically through the orifice according to a set of flow parameters.

2. The system of claim 1, further comprising a sensor component configured to obtain data describing the dissociation of the tissue sample into viable cells.

3. The system of claim 2, wherein the sensor component is an imaging component.

4. The system of claim 2, wherein the sensor component collects image data.

5. The system of claim 1, wherein the sample comprises a soft tissue, a neural tissue, a brain tissue, a cardiac tissue, a gastrointestinal tissue, a pancreatic tissue, or a live tissue.

6-7. (canceled)

8. The system of claim 1, wherein the flow parameters are associated with a sample type.

9. (canceled)

10. The system of claim 1, wherein the flow parameters are configurable by a user prior to or during dissociation of the tissue.

11-14. (canceled)

15. The system of claim 1, wherein the flow parameters comprise one or more of a flow rate through the microfluidic device, an oscillation frequency of flow through the microfluidic device, and/or a number of cycles of flow through the microfluidic device.

16-24. (canceled)

25. The system of claim 1, wherein said system is configured to process a sample having a weight from approximately 0.1 μg to approximately 1 mg.

26. The system of claim 1, wherein said system is configured to process a sample having a largest dimension that is approximately 50 μm to approximately 2 mm.

27-29. (canceled)

30. The system of claim 1, wherein the orifice has a width of from 10 μm to 500 μm, a length of from 10 μm to 1000 μm, and a height of from 200 to 500 μm.

31. The system of claim 1, wherein the microfluidic device comprises a channel that has a length of from 20 mm to 50 mm, a width of from 500 μm to 2 mm, and a height of from 50 μm to 500 μm.

32-38. (canceled)

39. The system of claim 1, comprising an apparatus comprising the programmable syringe pump, the sensor component, and an interface to accept the microfluidic device.

40-43. (canceled)

44. The system of claim 1, wherein the microfluidic device comprises a filter.

45-50. (canceled)

51. The system of claim 1, wherein the flow stress at the orifice is approximately 10 to 10⁴ dyne/cm².

52-54. (canceled)

55. The system of claim 1, wherein the system produces an output sample comprising at least 50% of the cells of the input tissue sample in a dissociated state.

56-59. (canceled)

60. The system of claim 1, wherein said system is configured to produce an output sample comprising cells, wherein at least 20% of said cells are viable.

61-65. (canceled)

66. A method for dissociating an input tissue sample into an output sample comprising dissociated viable cells, the method comprising:
a) providing a system according to claim 1;
b) providing a set of flow parameters to the programmable pump; and
c) providing an input sample for dissociation by the system.

67. The method of claim 66, comprising providing a first set of flow parameters to provide a first shear stress on the input tissue sample during cycles 1 to n and providing a second set of flow parameters to provide a second shear stress on the input tissue sample during cycles n+1 to m.

68-86. (canceled)

87. A device for dissociating an input tissue sample into an output sample comprising viable cells, the device comprising:
a microfluidic device comprising an orifice configured to induce a stress field on a tissue sample operably linked to a programmable pump configured to transport the tissue sample cyclically through the orifice according to a set of flow parameters.
